(19) United States
(12) Patent Application Publication
(10) Pub. No.: US 2013/0245387 A1
(43) Pub. Date: Sep. 19, 2013

(54) LOCATION BASED WIRELESS MEDICAL DEVICE

(71) Applicant: Maulin Dahyabhai Patel, Tuckahoe, NY (US)

(72) Inventor: Maulin Dahyabhai Patel, Tuckahoe, NY (US)

(73) Assignee: KONINKLIJKE PHILIPS ELECTRONICS N.V., EINDHOVEN (NL)

(21) Appl. No.: 13/883,770

(22) PCT Filed: Oct. 24, 2011
(86) PCT No.: PCT/IB11/54732

§ 371 Date: Jun. 3, 2013

Related U.S. Application Data
(60) Provisional application No. 61/410,992, filed on Nov. 8, 2010.

(51) Int. Cl. A61B 5/00

(52) U.S. Cl. CPC A61B 5/0015
USPC 600/300

(57) ABSTRACT

A personal area network (13) includes a plurality of wireless medical devices (12) which monitors physiological patient data and/or deliver therapy to the patient. The medical devices (12)each include a location management module (46) which controls a transceiver (40) to operate according to a operating profile associated with the geographical region in which the devices (12) currently reside. The operating profiles include at least one of transmission frequency, duty cycle, and maximum transmit power as mandated by local regulatory requirements. A wireless hub (10), as part of the personal area network (13), communicates with the wireless medical devices (12) and interfaces them with an infrastructure network (30). The hub (10) includes a location management module (46′) which receives a current geographical position and determines the corresponding geographical region and retrieves an operating profile(s) associated with the region. The hub advertises the operating profile to the wireless medical devices (12).

The present application relates to wireless devices. In particular, it relates to wireless sensor networks, such as body area networks (BANs) or patient area networks (PANs), which monitor a patient's physiological parameter and transmit a data regarding the sensed parameters to a control system.

Patients have traditionally been monitored using sensing units connected by wires to a base station. These wires inhibited the patient mobility and were labor intensive to install. To improve patient mobility, facilitate installation, and eliminate wire clutter, wireless sensing units have been developed. Certain patients require continuous monitoring of physiological parameters, such as ECG, SpO₂, blood pressure, blood sugar, or the like. Though well enough to move about the community, they were restricted to a hospital room, the hospital ward, a convalescent room, or their home to facilitate continuous monitoring of physiological parameters. To venture out of these areas, the patients would be unmonitored.

In order to continuously monitor patient physiological parameters without constraining their activities, it is desirable to be able to mount sensors on the body of the patient, which are light and compact as possible while also being capable of communicating wirelessly with each other and a base station. A body area network (BAN) includes multiple nodes which are typically sensors that can be either wearable or implantable in to the human body. The nodes monitor vital body parameters and/or movements, and communicate with each other over a wireless medium. The nodes can transmit physiological data from a body to a control unit from which the data can be forwarded, in real-time, to a hospital, clinic, or elsewhere over a local area network (LAN), wide area network (WAN), a cellular network, or the like.

Wireless technology provides convenient and unobtrusive connectivity between these devices. With the advances in energy efficient, reliable, low cost and high rate wireless technology, a variety of wireless consumer electronic devices have become integral part of our day to day life. Due to the portable nature of many of these devices, the likelihood of their proliferation beyond the regions in which they are authorized to operate has increased significantly. In particular, the wireless medical sensor devices designed to operate, in, on and around the human body to monitor and control various physiological parameters are expected to be carried globally by humans. Average human beings are unlikely to be aware of complex regulations governing the use of wireless devices. This means that the portable wireless devices must have provisions to reconfigure themselves to comply with local regulations.

The requirements for designing wireless BANs include providing convenient and unobtrusive connectivity between the nodes while maintaining energy efficient, reliable, low cost, high rate wireless connectivity and while adhering to geopolitical regulatory requirements regarding the use of the radio spectrum. The regulatory requirements mandate the compliance with technical requirements such as frequency-band usage, duty cycle limitations, bandwidth, maximum transmit power limitations, specific absorption rate, etc. However, these regulatory requirements are not harmonized worldwide. For example, 433.05-434.79 MHz band is designated as a license-free Industrial Scientific and Medical (ISM) band in Europe but not in US. On the other hand 902-928 MHz band is designated as a license-free ISM band in US but not in Europe. Therefore, wireless devices authorized to operate in one region may not be legally authorized to operate in another region. Even if the spectrum used by the device is available worldwide, the transmit power, duty cycle and other restrictions may be different in different regions thereby inhibiting the free movement of wireless devices across the border.

Due to the portable nature of many of these devices and the expected integration into day to day life, the likelihood that the wireless BAN devices being carried globally beyond the region in which they are authorized to operate should be planned for while understanding that the average patient is unlikely to be aware of local regulations governing the use of the wireless devices in that region. This poses a severe risk for the patient because non-compliant, unauthorized use of the wireless device can be detrimental to the wireless transmission functions between the devices which can interfere with sensing and/or therapeutic functions. There exists a need for wireless BANs and wireless portable devices to adjust wireless transmission parameters to meet local regulatory requirements for wireless transmission.

The present application provides a new and improved method and system for location based wireless patient monitoring and therapy delivery which overcomes the above-referenced problems and others.

In accordance with one aspect, a wireless medical device is presented. The wireless medical device includes at least one of a sensor which monitors physiological data of a patient and an actuator which delivers therapy to the patient. A wireless transceiver, which has a plurality of selectable operating parameters, transmits and/or receives information packets related to at least one of the monitored physiological data and delivered therapy. A location management module ascertains a current geographical position of the wireless medical device and determines a corresponding geographical region associated with the current geographical position. The location management module controls the wireless transceiver to operate according to one of the plurality of operating profiles based on the determined geographical region.

In accordance with another aspect, a method for wirelessly transmitting medical information is presented. The method includes at least one of monitoring physiological data of a patient and delivering therapy to the patient. Information packets related to at least one of the monitored physiological data and delivered therapy are wirelessly transmitted and/or received via a wireless transceiver. A current geographical position of the wireless medical device is ascertained and a corresponding geographical region associated with the current geographical position is determined. The wireless transceiver is controlled to operate according to one of the plurality of operating profiles based on the determined geographical region.

One advantage is that wireless medical devices maintain compliance to local regulatory requirements for wireless transmissions regardless of geographical location.

Still further advantages of the present invention will be appreciated by those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

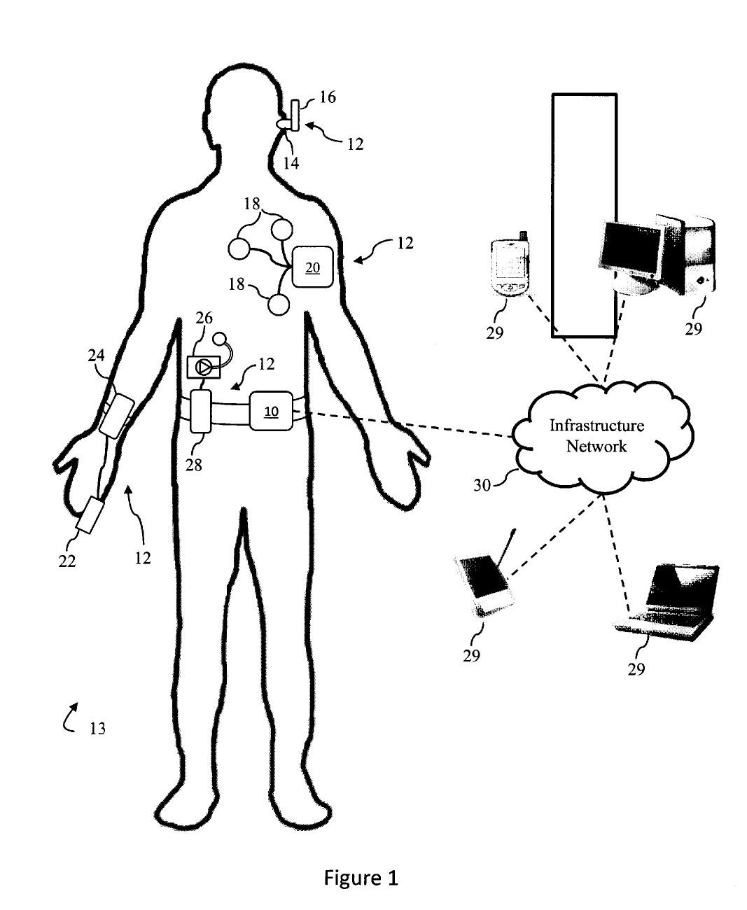

FIG. 1 is a diagrammatic illustration of a medical wireless network;

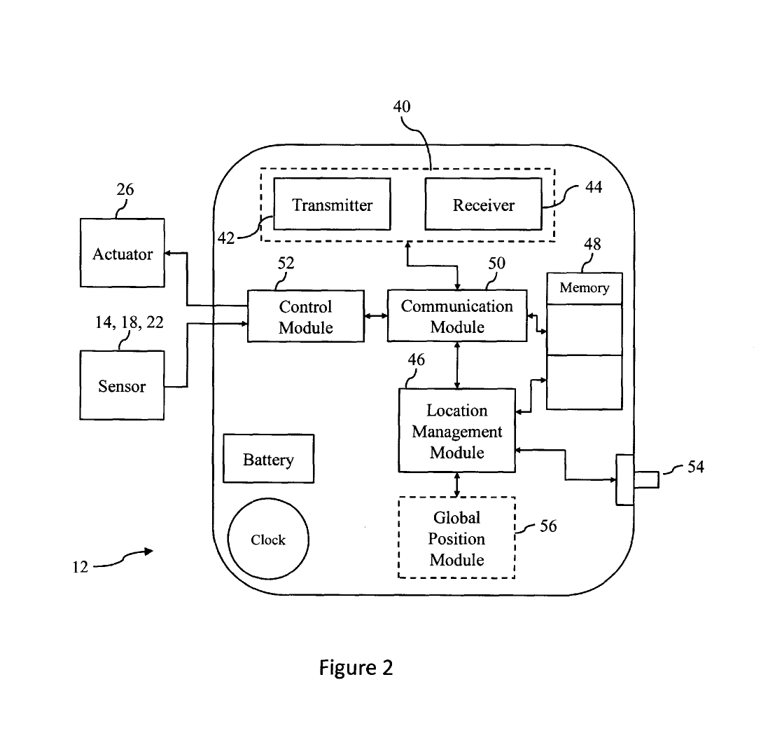

FIG. 2 is a detailed illustration of one of the wireless medical devices of FIG. 1;

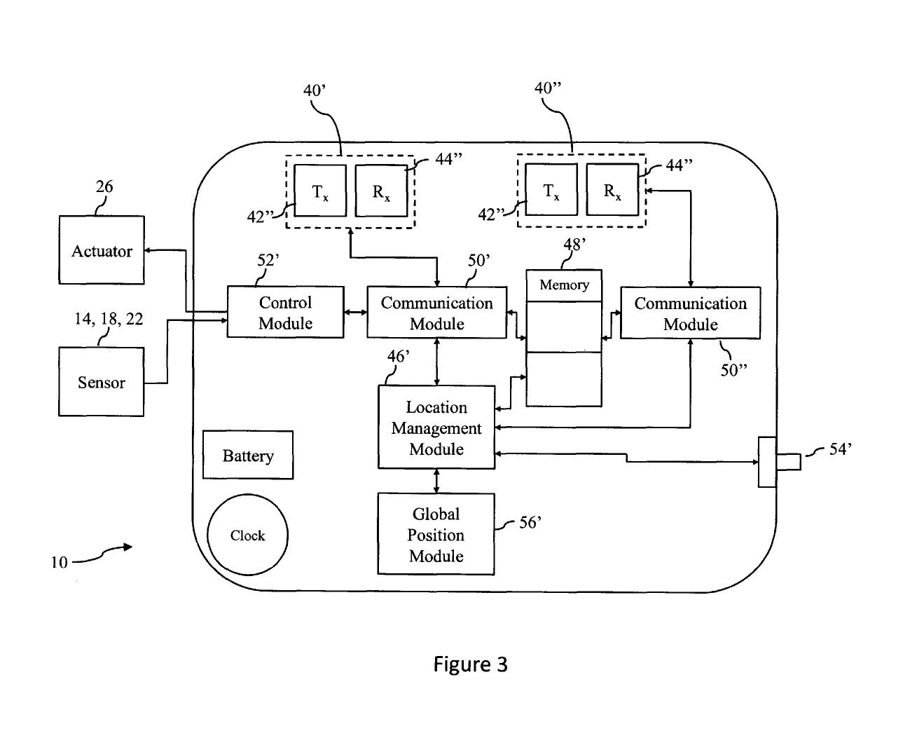

FIG. 3 is a diagrammatic illustration of the hub medical device of FIG. 1; and,

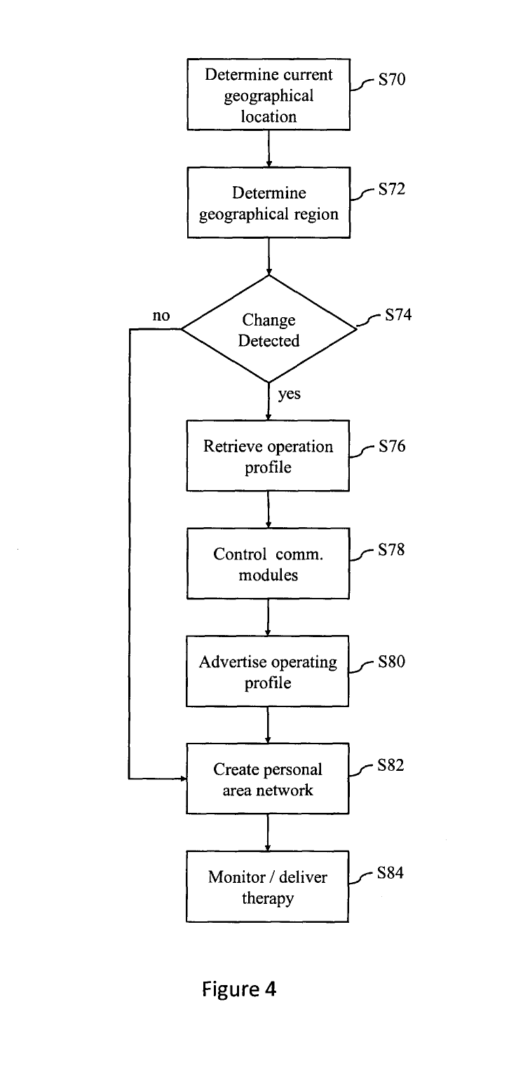

FIG. 4 is a flow chart illustrative of a method of operation.

With reference to FIG. 1, a plurality of wireless medical devices includes a hub medical device 10 and a plurality of other wireless medical devices 12, which form a personal area network (PAN) or a body area network (BAN) 13, arranged approximate to a patient's body for monitoring and recording various physiological parameters, administering therapy, or the like. The wireless medical devices 12 communicate wirelessly to the hub medical device 10. Various wireless medical devices 12 are contemplated, such as an inner-ear sensor 14 connected to an associated electronic module 16 which is disposed at least partially in the patient's ear to measure temperature, blood pressure, pulse rate, or the like. As another example, the wireless medical devices 12 can include an ECG monitor having a plurality of ECG sensors or electrodes 18 connected to an electronic module 20 which measures and interprets the sensed signals. As another example, an SpO2 sensor 22 senses blood oxygen and pulse rate, which are communicated by an associated electronics module 24. As another example, an infusion pump or other actuator 26 injects or otherwise dispenses medications into the patient's body under the control of electrical signals from an associated electrical module 28. Other wireless medical devices 12 which sense physiological parameters or deliver therapy includes pacemakers, hearing aids, vision aids, prosthetic limbs, artificial organs, and the like.

The wireless hub 10 conveys the received signals from the wireless medical devices 12 to other wireless medical devices 29, such as computer workstations, cellular phones, personal digital assistants, tablet computers, and the like, via an infrastructure network 30. It should be appreciated that the hub device can be a dedicated hub for the wireless medical device 12 or a multifunction device such as a cellular phone, personal digital assistant, tablet computer, and the like. Communications between the hub and the wireless network 30 can be via a wireless local area network (LAN) based on the IEEE 802.11 standards, via a wireless wide area network (WAN) such as a cellular network, via a campus area network (CAN), via a metropolitan area networks (MAN), via relatively high power RF transmissions, or the like.

The wireless medical devices 12 and the hub 10 may interact with one another in various configurations. For example, in a star network, each of the wireless medical devices 12 communicates directly with the hub medical device 10. The hub device receives acknowledgment signals or beacon signals from the devices 12 to, for example, synchronize the devices in anticipation of sending and receiving information packets, control signals, and the like, from the hub 10. In a mesh network, the devices 12 communicate directly with each other and the hub 10. Some of the devices 12 may communicate directly with the hub 10 or they may communicate with the hub 10 via other devices, such as computer, PDA's, mobile phone, or the like. These other devices may also communicate with other wireless medical devices 29 directly or via the infrastructure network 30 rather than via the hub 10.

With reference to FIG. 2, each wireless medical device 12 includes at least one of a sensor 14, 18, 22, which monitors physiological data of the patient or an actuator 26 which delivers therapy to the patient. The electronics module 20, 24, 28, associated with each sensor, actuator, or combination, includes a wireless transceiver 40 with a transmitter 42 and a receiver 44 which transmit and receive, respectively, information packets related to at least one of the monitored physiological data and/or the delivered therapy to/from at least one of the neighboring wireless medical device 12 and the wireless hub 10. Each wireless transceiver has as plurality of selectable operating parameters, such as frequency, duty cycle, bandwidth, maximum transmit power, and the like.

Each wireless medical device 12 includes a location management module 46. The location management module 46 ascertains a current geographical position of the wireless medical device 12 and determines a geographical region associated with the current geographical position. Tracking the geographical region, e.g. North America, Europe, Asia, South America, etc., of the wireless medical device ensures that the operation of transceiver 40 complies with the local regulatory requirements for wireless transmission for that region. Each geographical region is associated with at least one operating profile. Each operating profile defines a plurality of operating parameters for the wireless transceiver 40 which are associated with the geographical region. For example, an operating profile is defined for the United States of America (USA) which defines a transmission frequency, duty cycle, bandwidth and maximum transmit power for the transmitter 42 as mandated by the Federal Communications Commision (FCC). It should be appreciated that multiple operating profiles for a single geographical region are contemplated. Conversely, a single operating profile maybe associated with multiple geographical regions.

Once the geographical region is determined, the location management module 46 controls the wireless transceiver 40 to operate according to one of the operating profiles based on the determined geographical region. In one embodiment, the operating profiles are stored in a profile memory 48 of the wireless medical device 12. In another embodiment, the operating profiles are stored remotely and accessed wirelessly or received wirelessly by the transceiver 40. The operating profiles may be stored in a memory unit of the hub device 10. The hub device may transmit the operating profile or the wireless medical device 12 may request the appropriate operating profile. Alternatively, the hub device 10 and/or wireless medical device 12 may wirelessly access the stored operating profiles via the infrastructure network 30. In this arrangement, the operating profiles are stored on a computer readable medium that is part of a computer workstation or server which is part of a LAN, WAN, CAN, MAN, or the like.

A communication module 50 receives physiological information sensed by the sensor 14, 18, 22 via a sensor or actuator control module 52. The control module 52 also communicates with the actuator 26 to control its operation in accordance with received information packets. The communication module packages the sensed information and other transmission information such as acknowledgments, and the like, into information packets. The communication module controls the transceiver 40 to transmit the packets with an operating profile dictated by the location management module 46. It should be appreciated that the wireless medical device may include multiple transmitters 42 as part of the transceiver 40. Operating constraints may limit a single transmitter from operating at widely distinct frequencies. For example, a single transmitter may be capable of operating at the proposed 2.36 GHz MBAN frequency and the license free 2.4 GHz frequency. However, a second transmitter may be required to operate at the license free 433.05-434.79 MHz Industrial Scientific and Medical (ISM) band in Europe.

In one embodiment, each wireless medical devices 12 includes a user input 54, such as a switch, button, touch pad, input device, or the like, which is operated to input the corresponding geographical region to the location management module 46. As a switch, the user input 54 includes a plurality of user selectable position each of which is associated with at least one geographical region. As a button, the user may cycle through button presses of the user input 54 to select a corresponding geographical region or the user may select one of a plurality of buttons, each being associated with at least one geographical region. It should be appreciated that other user inputs 54, such as joystick, keypad, keyboard, touch-screen, touchpad, or the like, are also contemplated.

In another embodiment, each wireless medical device 12 includes an optional global positioning module 56 which determines a current geographical position using trilaterization of timing signals receive from global positioning satellites. The global positioning module 56 determines the current geographical location of the wireless medical device 12 and transmits the current geographical location to the location management module 46. From the current geographical position, the location management module 46 determines the geographical region in which the wireless medical device 12 currently resides.

With reference to FIG. 3, in another embodiment, the wireless medical device wirelessly receives the current geographical location from the hub device 10. The hub medical device 10 includes a first transceiver 40′ which communicates with the other wireless medical devices of the body network and a second transceiver 40″ which communicates with the infrastructure network 30. The wireless hub may be connected with a physiological data sensor and/or an actuator like the other wireless medical devices 12, or may function merely as a central controller or coordinator and for transferring physiological and/or therapy related information to and from the network 30. Similarly to the wireless medical devices 12, the hub 10 includes a location management module 46′ which ascertains a current geographical position of the hub 10 and determines an associated geographical region associated with the current geographical position. The location management module 46′ controls the wireless transceivers 40′, 40″ to operate according to one of the operating profiles based on the determined geographical region. The location management module 46′ receives the current geographical position from at least one of a user input 54′, global positioning module 56′, and via the infrastructure network 30. The user input 54′ and global positioning module 56′ function similar to that of the wireless medical devices 12. Using an input device, such as a switch (as illustrated), button, keyboard, joystick, keypad, touch-screen, touchpad, or other suitable input device, the user can select a geographical region.

As previously described, the hub 10 includes a profile memory 48′ which stores the operating profiles for the wireless medical devices 12 and their transceivers 40 and the transceivers 40′, 40″ of hub 10. Updated operating profiles which reflect changes in regulatory requirements can be obtain wirelessly from the infrastructure network 30 via the transceiver 40″. This is also advantageous if new frequency bands are introduced, for example the proposed MBAN band in the United States. Other changes include frequency band ranges, changes in duty cycle, changes in transmit power, or the like.

Communication modules 50′, 50″ receive and transmit information packets to/from the wireless medical devices 12 and the infrastructure network 30, respectively. The communications module 50′ controls the transceiver 40′ to transmit the packets with an operating profile dictated by the location management module 46′. Accordingly, The communications module 50″ controls the transceiver 40″ to transmit the packets with an operating profile dictated by the location management module 46′. If the hub unit 10 is connected with a sensor or actuator, then it also includes a sensor or actuator control module 52′.

With reference to FIG. 4, a method for selecting an operating profile and informing other wireless medical devices 12 is illustrated. In one embodiment, the hub device 10 acts as a master device, e.g. in a star network, which advertises the geographical region, current geographical position, or operating profile using information packets or beacon packets. The hub 10 determines the current geographical location (S70) from at least one of a user input 54′, global positioning module 56′, and via the infrastructure network 30. The location management module 46′ determines a geographical region (S72) in which the hub 10 and wireless medical devices 12 currently reside according to the determined current geographical location. If a change in the geographical region is detected (S74), the location management module 46′ retrieves the operation profile(s) associated with the geographical region (S76) from at least one of the profile memory 48′ or from a remote location via the infrastructure network 30. The location management module 46′ controls the communication modules 50′, 50″ (S78) to operate according to the operation profiles retrieved in step S76. The communication module 50′ controls the transceiver 40′ to transmit a beacon message to advertise the operating profile (S80) to the wireless medical devices 12. In another embodiment, the operating profile is embedded as part of an information packet, e.g. in a packet/frame transmission the operating profile can be embedded in the MAC address header or the PHY layer, and advertised as such. In a further embodiment, the geographical region or current geographical position rather than the operating profile is advertised to the wireless medical devices 12. After the wireless medical devices 12 have received the advertised operating profile or geographical region, a personal area network is created (S82) and monitoring of physiological data and therapy deliver may ensue (S84).

In another embodiment, the devices 10, 12 of the personal area network operate in a peer to peer configuration, e.g. in a mesh network. If one device detects a change in the geographical region which necessitates a change in the operating profile, then the device 10, 12 which detected that change advertises at least one of the geographical region and required operating profile. If the device includes a global position module 56, 56′, then it may also advertise the current geographical position. Upon hearing the advertised mode switch command, the location management module 46, 46′ of the neighboring wireless devices 10, 12 controls the transceivers 40, 40′, 40″ accordingly.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof

1. A wireless medical device, comprising:
at least one of a sensor which monitors physiological data of a patient and an actuator which delivers therapy to the patient;
a wireless transceiver which transmits and/or receives information packets related to at least one of the monitored physiological data and delivered therapy, the wireless transceiver having a plurality of selectable operating parameters;
location management module which ascertains a current geographical position of the wireless medical device, determines a corresponding geographical region associated with the current geographical position, and controls the wireless transceiver to operate according to one of the plurality of operating profiles based on the determined geographical region.

2. The wireless medical device according to claim 1, further including:
a profile memory which stores the plurality of operating profiles, each operating profile defines a plurality of operating parameters for the wireless transceiver which are associated with at least one geographical region.

3. The wireless medical device according to claim 1, wherein the operating parameters includes at least one of frequency, duty cycle, bandwidth and maximum transmit power.

4. The wireless medical device according to claim 1, further including:
a global positioning module which determines the current geographical location of the wireless medical device and transmits the current geographical location to the location management module.

5. The wireless medical device according to claim 1, further including:
a user input which is operated to input the corresponding geographical to the location management module.

6. The wireless medical device according to claim 1, wherein the location management module controls the wireless transceiver to transmit the determined geographical region and/or corresponding operating profile to a neighboring wireless medical device.

7. A wireless patient area network, comprising:
a plurality of wireless medical device according to claim 1; and
a wireless hub which interfaces the plurality of wireless medical device to an infrastructure network.

8. The wireless patient area network according to claim 7, wherein the wireless hub includes:
a wireless transceiver which communicates to the plurality of wireless medical devices to receive the monitored physiological data therefrom and to transmit an actuator control signal to control an actuator to deliver therapy thereto;
location management module which ascertains a current geographical position of the wireless hub and controls a wireless transceiver to transmit at least one of the current geographical position and the operating profiles for the current geographical position to at least one of the plurality of wireless medical devices.

9. The wireless patient area network according to claim 8, wherein the wireless hub further includes:
an infrastructure transceiver which communicates with the infrastructure network to transmit and receive information packets related to the wireless medical devices.

10. The wireless patient area network according to claim 9, wherein the wireless transceiver and the infrastructure transceiver are the same transceiver.

11. The wireless patient area network according to claim 8, wherein the wireless hub further includes:
a global positioning module which determines the current geographical location of the wireless hub and transmits at least one of the current geographical location and the operating profiles for the current geographical position to the location management module.

12. The wireless patient area network according to claim 9, wherein the location management module determines the current geographical position from the infrastructure network.

13. A method for a patient area network, comprising:
creating a wireless patient area network which includes a plurality of wireless medical devices according to claim 1 and at least one wireless hub; and
interfacing the plurality of wireless medical devices the wireless hub to an infrastructure network.

14. The method according to claim 13, further including:
determining a current geographical position of the wireless hub;
determining a corresponding geographical region according to the ascertained geographical position; and
controlling the wireless transceivers of the wireless medical devices to operate according to one of the plurality of operating profiles based on the determined geographical region.

15. A method for wirelessly transmitting medical information, comprising:
at least one of monitoring physiological data of a patient and delivering therapy to the patient;
via a wireless transceiver, wirelessly transmitting and/or receiving information packets related to at least one of the monitored physiological data and delivered therapy, the wireless transceiver having a plurality of selectable operating parameters;
ascertaining a current geographical position of the wireless medical device;
determining a corresponding geographical region associated with the current geographical position; and
controlling the wireless transceiver to operate according to one the plurality of operating profiles based on the determined geographical region.

16. The method according to claim 15, further including:
storing the plurality of operating profiles, each operating profile defining a plurality of operating parameters for the wireless transceiver which are associated with at least one geographical region;

17. The method according to claim 15, further including:
determining the current geographical location of the wireless medical device and transmitting the current geographical location; or
manually determining the corresponding geographical region and transmitting the corresponding geographical region to the location management module.

18. The method according to claim 15, wherein the operating parameters includes at least one of frequency, duty cycle, bandwidth and maximum transmit power.

19. The method according to claim 14, further including:
controlling the wireless transceiver to transmit the determined geographical region and/or corresponding operating profile to a neighboring wireless medical device.

20. A wireless medical device including a processor programmed to perform the method according to claim 13.
